(19) United States
(12) Patent Application Publication
(10) Pub. No.: US 2012/0089068 A1
(43) Pub. Date: Apr. 12, 2012

(54) Silver-Embedded Foam Dressing

(75) Inventor: Robert L. MCCLURE, JR., Elizabethton, TN (US)

(73) Assignee: WEBTEC Converting, LLC., Knoxville, TN (US)

(21) Appl. No.: 12/900,927

(22) Filed: Oct. 8, 2010

(51) Int. Cl. A61F 13/00

(52) U.S. Cl. 602/48

(57) ABSTRACT

A dressing that comprises a contact layer for contacting a wound, with the contact layer including silicone; a foam-based hydrophilic middle layer that carries a metal-based antimicrobial agent; and a backing layer that prevents moisture within the dressing from escaping into the air through evaporation. Silicone in the contact layer interacts with the wound to minimize the appearance of scar tissue. Moisture, either applied beforehand or from the wound exudate, travels through the porous contact layer into the foam middle layer. There, the moisture acts to release metal ions from controlled-release mechanisms, such as zeolites containing the metal ions. The ions then travel within the moisture into the wound, where they act to kill bacteria and other infectious microorganisms and to prevent infections from gaining a foothold. Many embodiments, for the metal-based antimicrobial agent, use silver ions or a combination of silver ions and other metal ions.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

The present invention relates to medicated, antimicrobial, or antibacterial dressings and more particularly to an antimicrobial and hydrophilic foam-based layered dressing impregnated with and carrying a metal-based antimicrobial agent.

2. Description of the Related Art

Dressings have long been known in the art for protecting and treating wounds or areas of the epidermis that display irritation or visible infection. (Hereinafter, “wounds” is understood to encompass a wide variety of skin injuries and irregularities, including lacerations and puncture wounds as well as, e.g., rashes and eczema.) In recent decades, medical practitioners have come to understand the benefits of including an antimicrobial or anti-infection agent in the dressing to prevent infection of the wound during treatment.

Scarring is a natural part of the healing process. Scar tissue consists mainly of protein collagen formed during the skin's process of wound repair. With the exception of very minor lesions, skin wounds following accidents, disease or surgery all result in some degree of visible scarring. Where the scar tissue is large or in a prominent position on the body, it can be readily apparent to a casual observer and embarrassing or otherwise troubling for the scarred person. It is therefore desirable to have a wound dressing that can help minimize the appearance of scarring during the healing process.

An open wound is at a heightened risk of infection throughout the healing process. In particular, microorganisms such as bacteria and fungi will attempt to establish themselves in the moisture of the exudate extruded from the wound during the healing process. Medical practitioners have discovered that certain metals and metallic compounds, and in particular silver ions, when delivered to a wound, can kill microorganisms within and on the surface of the wound and thereby help fight infection.

It is therefore desirable to have a wound dressing that can supply an antimicrobial agent, such as silver ions, to a wound while also helping the wound to heal in a way to reduce the appearance of scar tissue.

### BRIEF SUMMARY OF THE INVENTION

The present invention includes, in some embodiments, a dressing that comprises a contact layer for being placed directly on a wound, with the contact layer including silicone; a foam-based hydrophilic middle layer that carries a metal-based antimicrobial or anti-infection agent (hereinafter “antimicrobial agent”); and a backing layer that prevents moisture within the dressing from escaping into the air through evaporation. In some embodiments, the contact layer comprises a porous mesh of polyethylene fibers, with some or all of the fibers being coated with silicone. In some embodiments, the middle layer comprises a foam fabricated from a hydrophilic polyurethane or comparable material. The foam middle layer includes a metal-based antimicrobial agent that undergoes a controlled release when the middle layer comes into contact with moisture. In some embodiments, the backing layer comprises a thin film formed from a moisture-impermeable or non-porous material; this film forms a moisture-layer seal that acts as a moisture shield to keep moisture from the wound from escaping through the porous contact layer and the hydrophilic middle layer to be evaporated in the air on the other side of the dressing. Thus the backing layer helps to keep the wound from becoming too dry.

When the dressing is used, the contact layer is applied directly to a patient's skin, covering the wound. The silicone in the contact layer interacts with the wound to minimize the appearance of scar tissue. The dressing is either pre-wetted or applied dry to the wound. Moisture, either applied beforehand or from the wound exudate, travels through the porous contact layer into the foam middle layer. There, the moisture acts to release metal ions from controlled-release mechanisms, such as zeolites containing the metal ions. The ions then travel within the moisture into the wound, where they act to kill bacteria and other infectious microorganisms and to prevent infections from gaining a foothold. The backing layer helps to keep the wound from becoming too dry by inhibiting the evaporation of the moisture travelling between the wound and the dressing. Many embodiments, for the metal-based antimicrobial agent, use silver ions or a combination of silver ions and other metal ions.

The dressing according to some embodiments of the present invention thus provides a uniform and continuous release of antimicrobial ions while also helping to minimize the appearance of scar tissue.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view of one embodiment of the present invention;

FIG. 2 is an exploded view of the embodiment shown in FIG. 1;

FIG. 3 is a top-down view of the embodiment shown in FIGS. 1 and 2; and

FIG. 4 is a sectional view of the embodiment shown in FIGS. 1, 2, and 3.

### DETAILED DESCRIPTION OF THE INVENTION

The present invention includes, in some embodiments, a dressing that comprises a first layer or contact layer for contacting a wound, with the first layer including silicone; a foam-based hydrophilic middle layer that carries a metal-based antimicrobial or anti-infection agent (hereinafter “antimicrobial agent”); and a backing layer that prevents moisture within the dressing from escaping into the air through evaporation.

In some embodiments, the contact layer comprises a porous mesh of polyethylene fibers, with some or all of the fibers being coated with silicone. The middle layer comprises a foam fabricated from a hydrophilic polyurethane or comparable material. The foam middle layer includes a metal-based antimicrobial agent that undergoes a controlled release when the middle layer comes into contact with moisture. The backing layer comprises a thin film formed from a moisture-impermeable or non-porous material; this film forms a moisture-layer seal that acts as a moisture shield to keep moisture from the wound from escaping through the porous contact layer and the hydrophilic middle layer to be evaporated in the air on the other side of the dressing. When the dressing is used, the contact layer is applied directly to a patient's skin, covering the wound. The silicone in the contact layer interacts with the wound to minimize the appearance of scar tissue. The dressing is either pre-wetted or applied dry to the wound. Moisture, either applied beforehand or from the wound exudate, travels through the porous contact layer into the foam middle layer. There, the moisture acts to release metal ions from controlled-release mechanisms, such as zeolites containing the metal ions. The ions then travel within the moisture into the wound, where they act to kill bacteria and other infectious microorganisms and to prevent infections from gaining a foothold. The backing layer helps to keep the wound from becoming too dry by inhibiting the evaporation of the moisture travelling between the wound and the dressing. Many embodiments, for the metal-based antimicrobial agent, use silver ions or a combination of silver ions and other metal ions.

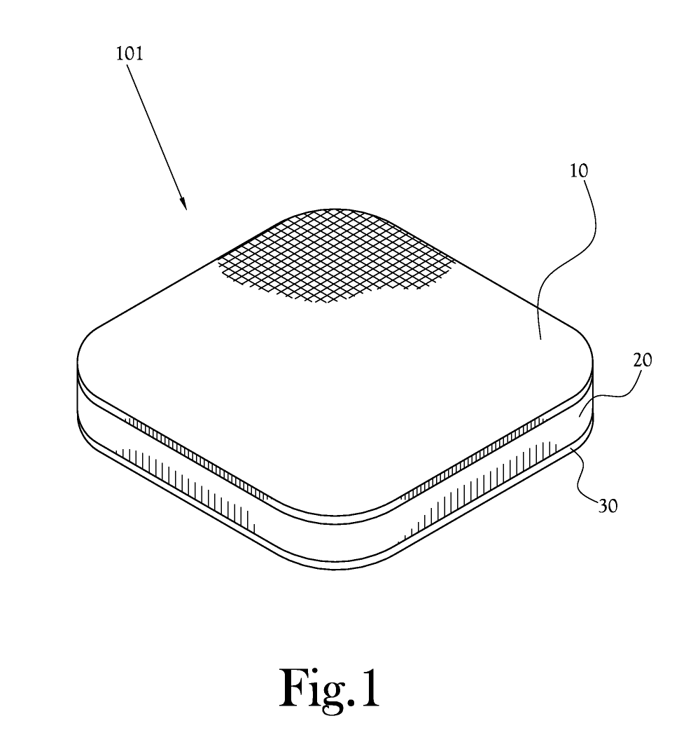

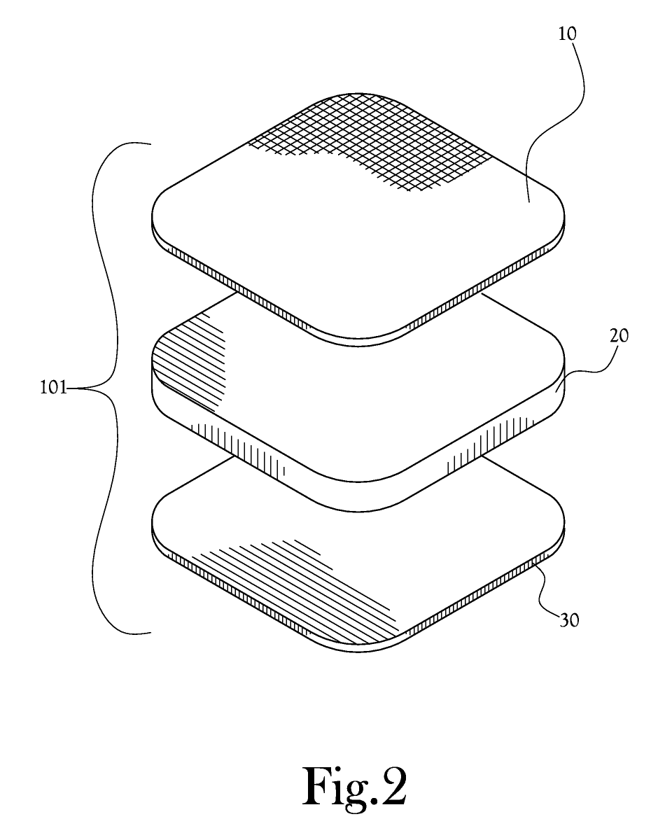

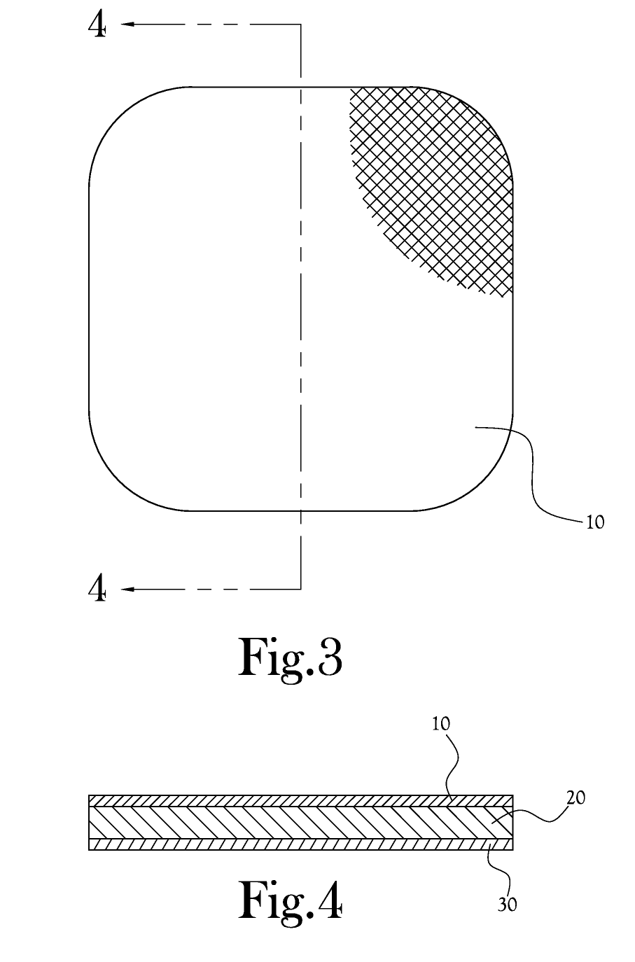

In one embodiment of the present invention, shown in FIGS. 1-4, a dressing 101 includes a contact layer 10 for contacting a wound, a foam-based middle layer 20 for carrying an anti-microbial agent, and a backing layer 30 for preventing the escape of moisture from the dressing 101 into the air.

In some embodiments, the contact layer 10 comprises a porous mesh of fibers wherein some or all of the fibers are coated in silicone. When the dressing is used on a person with a wound, the contact layer 10 is placed directly over the wound and comes into physical contact with the wound. The silicone in the contact layer 10 interacts with the wound to reduce the appearance of scar tissue. The contact layer 10 is hydrophobic, and thus moisture passes through the porous mesh of the contact layer 10 without being absorbed. In some embodiments, the contact layer 10 comprises an apertured Delnet® film fabricated from polyethylene. In some particular embodiments, the contact layer 10 comprises a silicone-coated Delnet® polyethylene film with apertures having an average diameter between approximately 300 microns and 500 microns.

In the some embodiments, the middle layer 20 comprises foam fabricated from a hydrophilic polyurethane. In some embodiments, other foam materials, such as latex foams, are used. In some embodiments, the foam middle layer 20 comprises an open cell foam material, in which the foam material contains pores forming an interconnected network that allows the relatively free movement of moisture within the foam. In some embodiments, the foam middle layer 20 comprises a Medisponge® foam. The foam middle layer 20 includes a metal-based antimicrobial agent that undergoes a controlled release when the middle layer 20 comes into contact with moisture. In some embodiments, the foam of the middle layer is fabricated from a fibrous substrate in which some or all of the fibers are coated in a zeolite carrying metal ions. In some embodiments, the foam middle layer 20 carries a silver salt with a moderate solubility in water, or some other salt which will release antimicrobial ions when encountering water. When the dressing 101 is in use on a person and encounters moisture, as from exudate from a wound, and moisture passes through the porous and meshy contact layer 10 and penetrates to the middle layer 20, where the moisture acts to release the antimicrobial metal ions from the foam. The ions then migrate through the porous, meshy contact layer 10 into the wound, where they kill microorganisms that cause infection. Moisture is necessary for most microbes and infectious organisms to thrive; with the present invention, the presence of that same moisture acts as a trigger to release the antimicrobial agent.

Some embodiments of the present invention include a backing layer 30. In some embodiments, the backing layer 30 is fabricated from a moisture-impermeable or non-porous material. The backing layer acts 30 as a moisture barrier or shield to keep moisture from the wound from escaping through the substrate layer to be evaporated in the air on the other side of the dressing. Thus the backing layer helps to keep the wound from becoming too dry.

In some embodiments, the backing layer 30 also includes a second antimicrobial agent that supplements the microbe-killing activity of the first antimicrobial agent in the substrate layer. In some embodiment, the second antimicrobial agent is silver magnesium phosphate or a zeolite impregnated with silver ions or a mixture of silver ions with zinc or copper ions. In these embodiments, when moisture penetrates the middle layer 20 and reaches the backing layer 30, the moisture releases the second antimicrobial agent from the backing layer; the second antimicrobial agent then migrates within the moisture through the middle layer 20 and the contact layer 10 into the wound. In this way, the second antimicrobial agent from the backing layer 30 supplements the antimicrobial agent carried in the substrate layer. In some embodiments, the backing layer 30 is a Delnet® mesh that carries one or more antimicrobial agents, such as silver magnesium phosphate or a zeolite impregnated with silver ions or a mixture of silver ions with zinc or copper ions.

Turning again to the foam middle layer 20, in some embodiments, the metal-based antimicrobial agent carried in the middle layer 20 includes antimicrobial ions carried in zeolites; in many embodiments, these antimicrobial ions are silver ions. Silver ions, when introduced into wounds, have been shown to have antimicrobial properties that are useful in preventing and fighting infection. In some embodiments, other antimicrobial metal ions are used, including sodium, copper, and zinc. In some embodiments, the middle layer carries a mixture of silver ions and copper ions. In some embodiments, the middle layer carries a mixture of silver ions and zinc ions. In some embodiments, the middle layer carries a mixture of silver ions and sodium ions.

In some embodiments, the silver ions or other ions are contained within controlled-release mechanisms, such as the zeolites described above, in order to provide a uniform and continuous ionic release. When the dressing is in use, the vast majority of the metal ions are not released at any one time but remain captive within the middle layer 20.

Where the antimicrobial agent used is a controlled-release silver-ion source, it has been found in tests that a minimum effective concentration of 63 ppm of silver in the dressing is necessary in order to achieve high effective bacterial control over a three-day use period. However, lower concentrations of silver or other ions are used in some embodiments, although these alternative embodiments with lower concentrations generally show less thorough or less robust antimicrobial results. For dressings with a minimum effective concentration of silver of 63 ppm or higher, ionic silver provides broad-spectrum antimicrobial inhibitory activity in the ppb to ppm range.

When used, many dressings embodying the present invention may be applied to a patient's skin either dry or pre-wet with de-ionized (DI) water or saline solution. For one embodiment of the present invention, multiple dressings comprising the same embodiment were tested for their silver elution over a 72-hour period, with set of dressings wetted with DI water and another set of dressings wetted with 0.9% saline solution. The results of these tests are summarized in Table 1.

TABLE 1
Elution Tests

| | Silver Content in | Average Silver Elution | Average % |
|---|---|---|---|
| Elutant | Dressing (ppm) | Measurement (ppm) | Eluted |
| DI Water | 63.2 | 35 | 0.055% |
| Saline | 63.2 | 75 | 0.119% |

As shown in Table 1, a wound dressing according to the present invention over a 72-hour period eluted an average of 0.055% of its silver content into DI water. Also, a wound dressing according to the present invention over a 72-hour period eluted an average of 0.119% of its silver content into 0.09% saline solution. Both elution percentages are small enough to not be toxic to the patient but still high enough to be effective at killing microbes and infectious organisms. Both test results confirm that the vast majority of the silver in the dressing is not released at any one time but remains captive within the middle layer of the dressing. A moderately higher level of silver is released into saline solution than into DI water. This finding is likely due to the presence of sodium chloride in the saline solution, as the chloride ion in the sodium chloride attracts and complexes with available silver ions to form silver chloride (which also has antimicrobial properties). Most human wound exudate contains some small percentage of sodium chloride, and so even when a dressing is applied dry to a patient's wound, it might be expected that the amount of silver released would be slightly higher than in tests were the elutant was DI water.

In some embodiments, the antimicrobial agent carried in the foam middle layer 20 includes a silver salt with a moderate solubility in water, such as silver sulfate, silver phosphate, silver citrate, silver acetate, silver carbonate, silver lactate, or silver magnesium phosphate, or a mixture of these salts. Some embodiments include other salts with ions that exhibit antimicrobial activity.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. An antimicrobial dressing comprising:
a meshy contact layer to contact a wound, said contact layer including silicone;
a hydrophilic foam middle layer in contact with said contact layer, said foam middle layer including a metal-based antimicrobial agent to permeate the meshy contact layer to contact the wound; and
a moisture-impermeable backing layer in contact with said middle layer opposite said contact layer, said backing layer configured to inhibit moisture from escaping said middle layer.

2. The antimicrobial dressing of claim 1 wherein said antimicrobial agent is selected from the group consisting of silver, sodium, copper, and zinc.

3. The antimicrobial dressing of claim 1 wherein said antimicrobial agent is a zeolite carrying metal ions, said metal ions selected from the group consisting of silver, sodium, copper, and zinc.

4. The antimicrobial dressing of claim 1 wherein said antimicrobial agent is a zeolite carrying a first metal ion and a second metal ion, wherein said first metal ion is silver and said second metal ion is selected from the group consisting of sodium, copper, and zinc.

5. The antimicrobial dressing of claim 1 wherein said foam middle layer comprises fibers coated with zeolites carrying metal ions.

6. The antimicrobial dressing of claim 5 wherein said fibers are fabricated from polyurethane.

7. The antimicrobial dressing of claim 1 wherein said antimicrobial agent is a silver salt.

8. The antimicrobial dressing of claim 7 wherein said silver salt is selected from the group consisting of silver sulfate, silver phosphate, silver citrate, silver acetate, silver carbonate, silver lactate, and silver magnesium phosphate.

9. A foamy laminar antimicrobial dressing comprising:
a fibrous first layer to contact a wound, said first layer including fibers coated with silicone;
a second layer including a foam fabricated from fibers coated with zeolites, said zeolites carrying a metal ion selected from the group consisting of silver, sodium, copper, and zinc; and
a hydrophobic third layer in contact with said second layer opposite said first layer to inhibit the passage of moisture from the second layer to the air.

10. The foamy laminar antimicrobial dressing of claim 9 wherein said zeolites are carrying a mixture of silver ions and copper ions.

11. The foamy laminar antimicrobial dressing of claim 9 wherein said zeolites are carrying a mixture of silver ions and sodium ions.

12. The foamy laminar antimicrobial dressing of claim 9 wherein said zeolites are carrying a mixture of silver ions and zinc ions.

13. The foamy laminar antimicrobial dressing of claim 9 wherein said hydrophobic third layer is carrying a second antimicrobial agent.

14. The foamy laminar antimicrobial dressing of claim 13 wherein said second antimicrobial agent includes a zeolite carrying silver ions.

15. The foamy laminar antimicrobial dressing of claim 13 wherein said second antimicrobial agent includes a zeolite carrying a mixture of silver ions and zinc ions.

16. The laminar antimicrobial dressing of claim 13 wherein said second antimicrobial agent includes silver magnesium phosphate.

17. An antimicrobial dressing comprising:
a hydrophobic meshy contact layer to contact a wound, said contact layer including silicone; and
a moisture-permeable fibrous foam layer, said foam layer including fibers coated with zeolites, said zeolites being carrying a metal ion selected from the group consisting of silver, sodium, copper, and zinc.

18. The antimicrobial dressing of claim 17 wherein said zeolites are carrying a first metal ion and a second metal ion, wherein said first metal ion is silver and said second metal ion is selected from the group consisting of sodium, copper, and zinc.
